(19) United States
(12) Patent Application Publication
(10) Pub. No.: US 2021/0314729 A1
(43) Pub. Date: Oct. 7, 2021

(54) SYSTEMS AND METHODS FOR MONITORING SOCIAL DISTANCING AND CONTACT TRACING

(71) Applicant: Triax Technologies, Inc., Norwalk, CT (US)

(72) Inventors: Justin J. Morgenthau, South Windsor, CT (US); Ryan Edwin Downing, Philadelphia, PA (US); Thomas Steven Ludorf, Englewood, NJ (US); Ian Michael Ouellette, Norwalk, CT (US)

(73) Assignee: Triax Technologies, Inc., Norwalk, CT (US)

(21) Appl. No.: 17/223,212

(22) Filed: Apr. 6, 2021

Related U.S. Application Data
(60) Provisional application No. 63/005,855, filed on Apr. 6, 2020.

(51) Int. Cl. H04W 4/02; A61B 5/00; H04B 17/318

(52) U.S. Cl. CPC H04W 4/023; A61B 5/6803; A61B 5/0022; A61B 5/7405; H04W 84/18; A61B 2560/0209; A61B 2560/0223; A61B 2503/20; H04B 17/318

(57) ABSTRACT

In some aspects, a system comprises a first wearable sensor configured to be attached to a first user, the first wearable sensor adapted to periodically transmit a message, the message containing a sensor identifier that uniquely identifies the first wearable sensor, wherein the first wearable sensor is configured to listen for one or more messages transmitted by one or more wearable sensors attached to other users, and wherein the first wearable sensor is adapted to determine a signal strength of a received message, and determine is the signal strength is greater than a threshold, issue an alert, and a gateway adapted to communicate with the first wearable sensor.

### CROSS REFERENCE TO RELATED APPLICATIONS

This application is a Non-Provisional of Provisional (35 U.S.C. § 119(e)) of U.S. Provisional Patent Application Ser. No. 63/005,855, filed Apr. 6, 2020, entitled “SYSTEMS AND METHODS FOR MONITORING SOCIAL DISTANCING AND CONTACT TRACING,” which is incorporated herein by reference in its entirety.

### BACKGROUND

There are many challenges facing essential industries during the current COVID-19 pandemic. Employers are asking employees to practice “social distancing,” which in the workplace typically involves maintaining a distance of at least six feet from other workers. When a worker has tested positive for COVID-19, “contact tracing” becomes critical, where Centers for Disease Control and Prevention (CDC) guidelines require identification of everyone the infected worker came into contact with at less than a six feet distance for more than 15 minutes in a given day, for the past 14 days. These subjects are then directed to self-quarantine for 14 days.

### SUMMARY

In some aspects, systems and methods are described herein for monitoring and enforcing social distancing and/or contact tracing. In some embodiments, such systems and methods are based on a system for monitoring safety at a construction site (e.g., a jobsite monitoring system available commercially, such as the Spot-r® system available from Triax Technologies, Norwalk, Conn., or another suitable system). During the ongoing COVID-19 crisis, there is a desire to remind construction workers to maintain appropriate distance from each other (e.g., six feet by CDC guidelines) and also to provide the capability for contact tracing should a worker test positive for the virus. This system has potential applications in many other types of businesses including manufacturing and even office settings. This system may be equally applicable to other types of viruses or infectious diseases where social distancing and/or contact tracing may be desirable.

Some aspects relate to a system comprising a first wearable sensor configured to be attached to a first user, the first wearable sensor adapted to periodically transmit a message, the message containing a sensor identifier that uniquely identifies the first wearable sensor, wherein the first wearable sensor is configured to listen for one or more messages transmitted by one or more wearable sensors attached to other users, and wherein the first wearable sensor is adapted to determine a signal strength of a received message, and based on determining that the signal strength is greater than a threshold, issue an alert; and a gateway adapted to communicate with the first wearable sensor.

In some embodiments, the first wearable sensor is configured to communicate with one or more wearable sensors attached to other users in a low power mode, and the first wearable sensor is configured to switch to a high power mode in order to communicate with the gateway, the high power mode using more power than the lower power mode and increasing communication range for the first wearable sensor.

In some embodiments, the first wearable sensor comprises a transmitter adapted to communicate with the one or more wearable sensors attached to other users using a sub-GHz radio band when in the low power mode.

In some embodiments, the gateway is adapted to capture contact session data from one or more wearable sensors including the first wearable sensor.

In some embodiments, the first wearable sensor is adapted to generate contact session data, the contact session data identifying at least one contact session set of information comprising a time at which the contact occurred, a duration of the contact, and an identifier of a sensor which came into proximity of the first wearable sensor.

In some embodiments, the first wearable sensor generating the contact session data includes the first wearable sensor converting ping data from the one or more wearable sensors attached to other users into the contact session data, including the at least one contact session set of information comprising the time at which the contact occurred, the duration of the contact, and the identifier of the sensor which came into proximity of the first wearable sensor.

In some embodiments, transmitting the contact session data to the gateway uses less bandwidth than transmitting the ping data from the one or more wearable sensors attached to other users.

In some embodiments, the first wearable sensor and the gateway each store a calibration value to include in a packet, and wherein a receiving device adds the calibration value from the packet to a measured Received Signal Strength Indicator (RSSI) value for the packet to determine a compensated RSSI value for the packet, the compensated RSSI value being compared to one or more thresholds to determine range.

In some embodiments, the gateway serves as a calibration reference device, and wherein the calibration value of the first wearable device is determined by transmitting, from the first wearable device, a calibration request to the calibration reference device; measuring, at the calibration reference device, an RSSI value of the calibration request; transmitting, from the calibration reference device, a response including the measured RSSI value of the calibration request and a calibration value of the calibration reference device; measuring, at the first wearable device, an RSSI value of the response; and computing, at the first wearable device, the calibration value of the first wearable device using the RSSI value of the response, the RSSI value of the calibration request, and the calibration value of the calibration reference device.

In some embodiments, the first wearable device and the gateway are configured to perform multipath interference mitigation, including exchanging packets on three or more different channels located at a low-end, middle and/or high-end of a frequency band; obtaining three or more RSSI measurements corresponding to each of the three or more different channels; and determining a characteristic RSSI value based on the three or more RSSI measurements.

In some embodiments, the first wearable sensor is adapted to sound an audible alert.

In some embodiments, the first wearable sensor is adapted to sound a progressive alert based at least in part on the duration of proximity to one or more wearable sensors attached to other users.

In some embodiments, the first wearable sensor is adapted to attach to a hard hat of the first user.

In some embodiments, the first wearable sensor is configured to attach to a lanyard of the first user.

In some embodiments, the sensor comprises an alert indicator and is adapted to activate the indicator in response to determining that the signal strength exceeded the threshold.

In some embodiments, a cloud-based storage system for storing contact session information communicated by the first wearable sensor.

In some embodiments, the threshold signal strength is correlated with a distance between the first wearable sensor and at least one of the one or more wearable sensors attached to other users.

Some aspects relate to a system comprising a plurality of wearable sensors configured to be attached to respective ones of a plurality of users; at least one gateway adapted to capture contact session data from the plurality of wearable sensors, the contact session data including contact events that identify contact that has occurred between respective pairs of the plurality of wearable sensors; and a management system adapted to store the contact session data from the plurality of wearable sensors.

In some embodiments, each one of the plurality of wearable sensors are adapted to record contact session data, the data identifying at least one contact session set of information comprising a time at which the contact occurred, a duration of the contact, and an identifier of a different wearable sensor which came into a proximity of at least one of the plurality of wearable sensors.

In some embodiments, each one of the plurality of wearable sensors comprises a transmitter adapted to communicate messages using a sub-GHz radio band.

In some embodiments, each of the plurality of wearable sensors communicates with other ones of the plurality of wearable sensors via the sub-GHz radio band.

In some embodiments, the each of the plurality of wearable sensors communicates with the gateway using the sub-GHz radio band.

In some embodiments, the gateway comprises a cellular communication component, and the gateway is adapted to communicate contact session data to the management system over the cellular communication component.

In some embodiments, the gateway comprises a wired communication component, and the gateway is adapted to communicate contact session data to the management system over the wired communication component.

In some embodiments, the gateway comprises a wireless communication component, and the gateway is adapted to communicate contact session data to the management system over the wireless communication component.

In some embodiments, the plurality of wearable sensors do not include GPS capability.

In some embodiments, the plurality of wearable sensors do not include cellular communication capabilities.

Still other aspects, embodiments, and advantages of these exemplary aspects and embodiments, are discussed in detail below. Moreover, it is to be understood that both the foregoing information and the following detailed description are merely illustrative examples of various aspects and embodiments and are intended to provide an overview or framework for understanding the nature and character of the claimed aspects and embodiments. Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment,” “at least one embodiment,” “this and other embodiments,” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this disclosure, but are not intended as a definition of the limits of a particular embodiment. The drawings, together with the remainder of the disclosure, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1A shows definitions according to the Centers for Disease Control and Prevention (CDC);

FIG. 1B is an exemplary diagram of subjects as defined according to the Centers for Disease Control and Prevention (CDC);

FIG. 1C is another exemplary diagram of subjects as defined according to the Centers for Disease Control and Prevention (CDC);

FIG. 2 shows definitions according to the New York State Department of Health (NY DOH) regarding individuals corresponding to contact tracing;

FIG. 3 shows an exemplary wearable device, according to various aspects of the embodiments described herein;

FIG. 4 shows an exemplary wearable system, according to various aspects of the embodiments described herein;

FIG. 5 shows an exemplary block diagram of a system capable of implementing various aspects of the embodiments described herein;

FIG. 6 shows exemplary gateways, also referred to as Cloud Pods herein, at an exemplary site, according to various aspects of the embodiments described herein;

FIG. 7 shows an exemplary control flow of a wearable device, according to various aspects of the embodiments described herein;

FIG. 8A shows an exemplary contact session processing control for a wearable device, according to various aspects of the embodiments described herein;

FIG. 8B shows an exemplary contact session state machine for a wearable device, according to various aspects of the embodiments described herein;

FIG. 9A is an exemplary signal strength range of device-to-device communication using a low power signal, according to various aspects of the embodiments described herein;

FIG. 9B is an exemplary signal strength range of device-to-gateway communication using a high power signal, according to various aspects of the embodiments described herein;

FIG. 10 shows results from a reviewal of performance, according to various aspects of the embodiments described herein;

FIG. 11 shows a flowchart of an exemplary process of determining distance using a calibration value, according to various aspects of the embodiments described herein;

FIG. 12 shows a flowchart of an exemplary process performed by a device and a calibration reference device to calibrate the device, according to various aspects of the embodiments described herein;

FIG. 13 shows an exemplary calibration reference and a device being calibrated, according to various aspects of the embodiments described herein;

FIG. 14 shows an exemplary calibration reference and a device being calibrated, according to various aspects of the embodiments described herein;

FIG. 15 shows a flowchart of an exemplary process for a device being calibrated, according to various aspects of the embodiments described herein;

FIG. 16 shows a flowchart of an exemplary process for a calibration reference device, according to various aspects of the embodiments described herein; and

FIG. 17 shows a flowchart of an exemplary multipath interference mitigation method, according to various aspects of the embodiments described herein.

### DETAILED DESCRIPTION

The current state-of-the-art in contact tracing involves interviews, during which infected individuals are asked to recall whom they were in contact with for the last 14 days. The inventors have appreciated that there is significant room for a technological improvement here, as incompleteness can result in further infections and overestimating contact results in unnecessary lost work. It is worth noting that South Korea was able to get COVID under control much faster than any other nation due in part to their effective use of cell phone data for contact tracing. Unfortunately, it is appreciated that a complete and universal cell phone based solution is not feasible in the US at present.

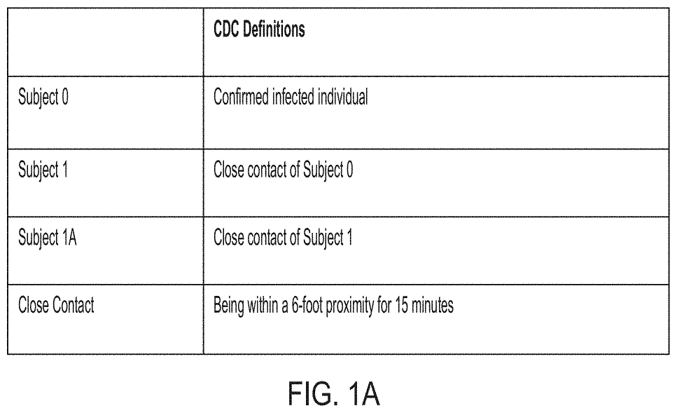

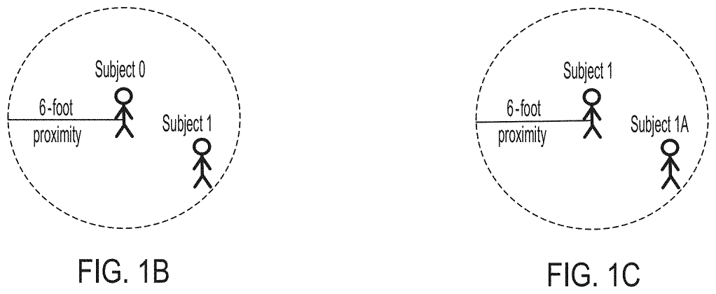

FIG. 1A shows definitions according to the Centers for Disease Control and Prevention (CDC). According to the CDC, an individual who is confirmed to be infected is considered a ‘Subject 0.’ An individual in close contact to ‘Subject 0’ is a ‘Subject 1’ and an individual in close contact to ‘Subject 1’ is a ‘Subject 1A,’ where close contact is defined by the CDC as being within a six foot proximity for 15 minutes or longer. FIG. 1B and FIG. 1C show exemplary diagrams of subjects as defined according to the Centers for Disease Control and Prevention (CDC). For example, FIG. 1B shows exemplary infected individual ‘Subject 0’ with another individual within a six foot proximity. If the individual remains within a six foot proximity to ‘Subject 0,’ the individual would become ‘Subject 1.’ FIG. 1C shows exemplary ‘Subject 1’ with another individual within a six foot proximity. If the individual remains within a six foot proximity to ‘Subject 1,’ the individual would become ‘Subject 1A.’

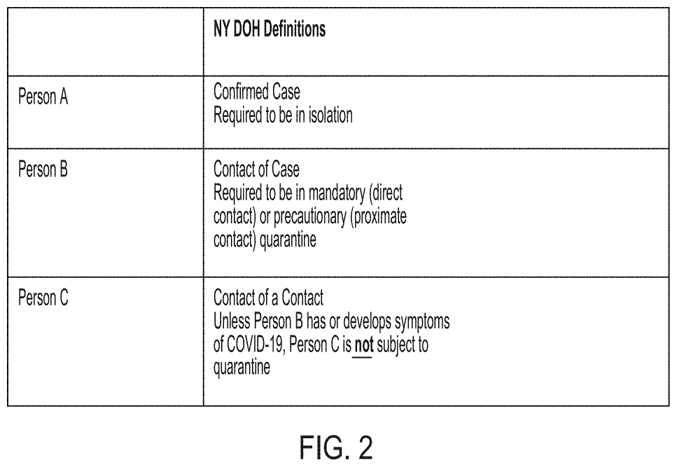

FIG. 2 shows definitions according to the New York State Department of Health (NY DOH) regarding individuals corresponding to contact tracing. According to the NY DOH, an individual who has contracted a confirmed case is considered a ‘Person A’ and according to guidelines set forth by the NY DOH, is required to be in isolation. According to the NY DOH, an individual who is a contact of ‘Person A’ is considered a ‘Person B’ and according to guidelines set forth by the NY DOH, is required to be in mandatory (direct contact) or precautionary (proximate contact) quarantine. A contact of a ‘Person B’ is considered a ‘Person C’ and unless ‘Person B’ has or develops symptoms of COVID-19, Person C is not subject to quarantine, according to guidelines set forth by the NY DOH.

Contact tracing is the process of identifying who an individual, with a confirmed case of COVID-19 (or other virus), came in contact with over a period of time. The CDC, European Centre for Disease Prevention and Control (ECDC), and NY DOH all cite six feet of social distancing as a key effort in flattening the curve and mitigating the spread of COVID-19. The ECDC further classifies by time of exposure with a key threshold of >15 minutes in a day within a six foot proximity. Some aspects relate to maintaining social distancing and help with contact tracing to identify “Subject 1/Person B” candidates who have been in contact with “Subject 0/Person A” to help target and prioritize, but not replace traditional mechanisms of confirmation. This can be done, for example, by the passive collection of worker interactions to identify potential close contact interaction should an individual test positive and also by providing active feedback to the worker, in the form of a visual and audible alarm, so individuals know when to adjust their current distance to a proper social distance (outside of a six foot proximity).

### Wearable Device Hardware

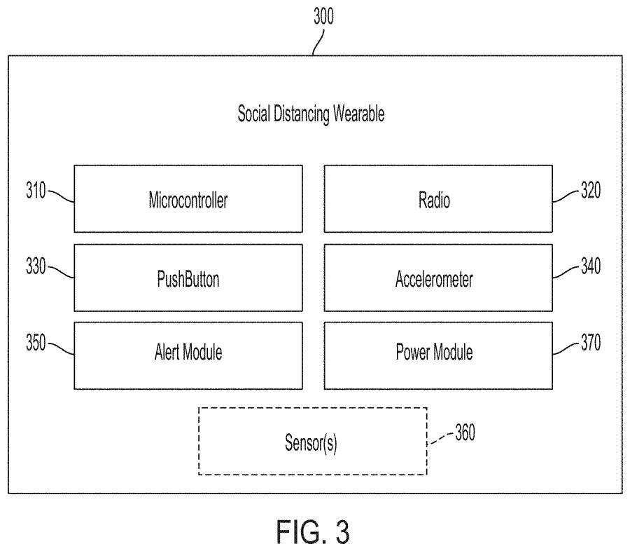

According to some aspects, a wearable device is provided. For example, FIG. 3 shows an exemplary wearable device. A wearable device may comprise any combination of, for example, a microcontroller 310, a radio 320, a pushbutton 330, an accelerometer 340, an alert module 350 and/or a power module 370.

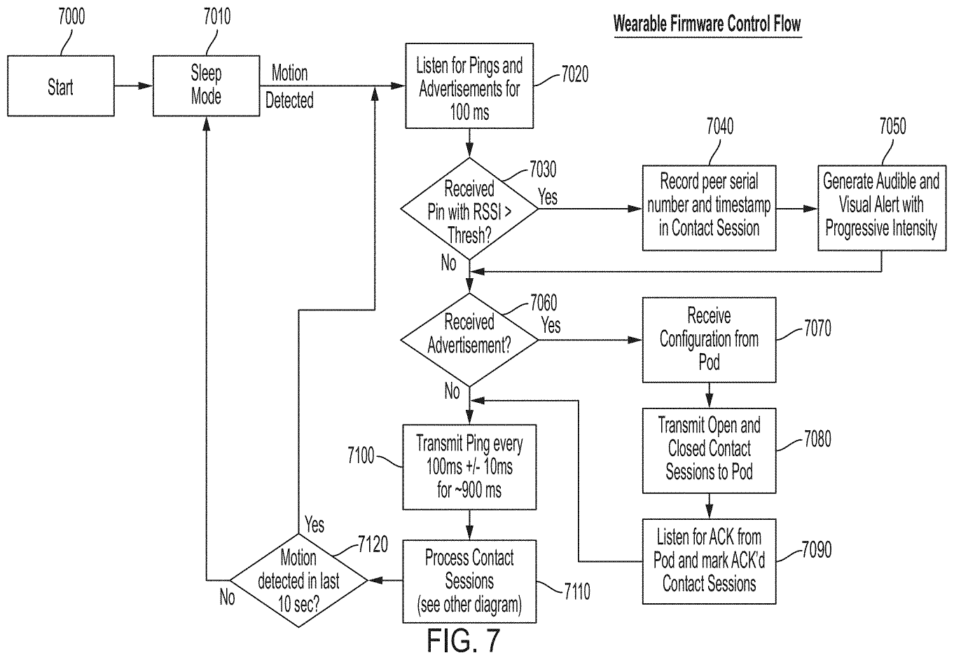

The accelerometer 340 may be used to determine movement of a wearer of the wearable device. For example, the microcontroller 310 may be communicatively coupled to at least one accelerometer adapted to detect motion of the piece of equipment. In some embodiments, the accelerometer 340 may be used to put the wearable device in a sleep mode (e.g., as described further in relation with FIG. 7) if data from the accelerometer indicates that a wearable is idle.

In some embodiments, the wearable does not include cellular communication capabilities. In some embodiments, the wearable comprises a transmitter adapted to communicate messages using a sub-GHz radio band.

The alert module 350, may be configured to alert the wearer of the wearable using audio and/or visual cues. For example, the alert module may comprise one or more light-emitting diodes (LEDs) to flash and create a visual alert. As another example, the alert module may include a speaker to create sound as an auditory alert. The alert module 350 may be configured to emit an audible social distancing alert and/or flash a red LED, in real time, from the wearable device when an interaction with another wearable device within a predetermined distance or range is determined. For example, the range may be six feet to 10 feet from another wearable, or another suitable distance. In some embodiments, the alert emits a warning beep for timely distance correction, but the alarm increases in intensity if distance is not corrected. In some embodiments, the wearable device is used to perform only contact tracing reports, and the alert module does not emit a flashing red light or an audible alert or another suitable alert.

As described here, the wearable device may also include a pushbutton 330. In some embodiments, the pushbutton 330 may be operated to turn an alert off. In some embodiments, the wearer of the wearable device may have the ability to silence an active alert with a press of the pushbutton. In some embodiments, a single press may disable an alert for a first predetermined period of time (e.g., 3 minutes). In some embodiments, a double press of the push button may disable the alert for a second predetermined period of time longer than the first predetermined period of time (e.g., 15 minutes). In some embodiments, the predetermined periods of time may be configured by a user of the system, the wearer and/or another suitable entity. Disabling the alarm by pressing the pushbutton may not disable the ability for contact tracing and data from a contact session may still be collected.

In some embodiments, the power module 370 may include a battery and/or a battery pack. For example, the power module may include a rechargeable battery. In some embodiments, the power module includes a power regulator. For example, the microcontroller 310 may be connected to the power regulator in order to draw power from the battery.

In some embodiments, the wearable device may be part of a wearable system. In some embodiments, the wearable system may have one or more attachments that are used to affix the device to the wearer.

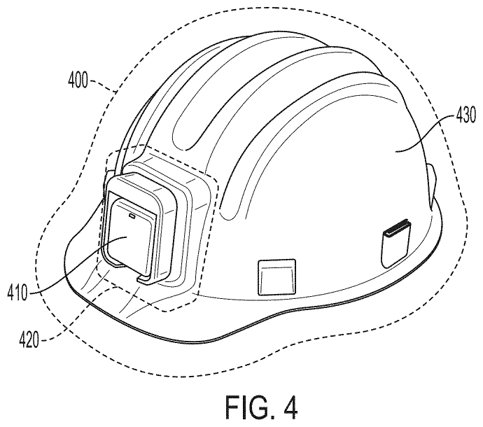

For example, in some configurations, the system comprises a wearable device for each worker mounted to the front of a hardhat. In some embodiments, the wearable may snap into a fixture which is mounted to the front of the hardhat, although direct mounting via Velcro strips, a clamp to the brim, and/or a strap around the hardhat, or other adhesive method is also possible, or the attachment may use other suitable methods. For example, FIG. 4 is an exemplary wearable system. The system 400 has a hardhat 430 with wearable device 410 and fixture 420 into which the wearable device 410 may be mounted on or snapped into. In some configurations, the fixture (e.g., fixture 420) may include a new single-piece injection-molded plastic part which would be attached using adhesive tape or Velcro strips. In some embodiments, the wearable device itself can be assembled without the metal belt-clip so that the wearable can sit flush to the hardhat. Mounting to the hardhat may provide uniform directional coverage, which increases the accuracy and consistency of Received Signal Strength Indicator (RSSI) measurements and ranging estimates.

In some embodiments, the wearable devices can be configured through firmware to communicate directly to each other without the need for a wireless mesh network or any gateways during normal operation.

In some embodiments, the wearable could also be worn around the neck (e.g., on a lanyard), centered on the chest. This can have some effect on accuracy on ranging behind the wearer but can still be largely effective, as compared to for example mounting on the hardhat.

According to some embodiments, the wearable system may have one or more additional sensors 360, which may include a sensor used to determine whether the sensor is coupled to the user. For example, the wearable may include an internal mechanism such that, when clipped to the user, a switch is activated. The switch may be used alone or in conjunction with the other sensor(s) 360 (e.g., a proximity sensor) to determine whether the sensor is coupled to the user.

In some embodiments the battery life of the wearable device may be expected to be in the range of 3-5 months depending on usage. One advantage of using specialized hardware that has a low energy use is that the device need not be recharged over long periods, does not need to be removed, etc. In some embodiments, the device may not require Global Positioning System (GPS) capability which may lead to longer battery life and user privacy. In some embodiments, the wearable device is a piece of hardware reprogrammed to perform methods described herein. For example, one or more components of a wearable device, such as those described in commonly owned U.S. Patent Application Publication No. 2017/0270462, may be reprogrammed to perform the methods described herein. In another example, hardware having the components described in relation with FIG. 3 and/or other components may be reprogrammed.

### Device System

As described herein, the wearable device can be a wearable device that has been reprogrammed to have different firmware. In some embodiments, the firmware for these reprogrammed devices may be different from the firmware previously present on the wearable device. In some embodiments, reprogramming the wearable device may simply include adding new functionality to pre-existing firmware.

In some embodiments, the reprogrammed wearable devices do not participate in a wireless mesh network. For example, rather than participating in a wireless mesh network, each wearable device may periodically transmit a message (e.g., a packet) containing a serial number identifier, or another suitable piece of information, to identify the wearable device a few times per second and listen for identifier packets when not transmitting.

The wearable device (e.g., device 300), may be used with other devices in a system to monitor and record, for example, interactions between people. In some embodiments, the wearable device may interact with other wearable devices using device-to-device signaling and the wearable device may also interact with a gateway using device-to-gateway signaling. In some embodiments, the gateway may be cellular and may not depend on client WiFi or Internet. In some embodiments, the wearable device may not be tracked once outside of a gateway's range and may not use GPS. In some embodiments, the gateway may be the Cloud Pod described herein.

In some embodiments, the gateway may include a wired communication component (e.g. Ethernet, etc.), and the gateway may be adapted to communicate contact session data to the management system over the wired communication component. Alternatively or additionally, the gateway may include a wireless communication component (e.g. WiFi, etc.), and the gateway may be adapted to communicate contact session data to the management system over the wireless communication component.

In some embodiments, a gateway may be cellular and further include wired (e.g. Ethernet, etc.) and/or wireless (e.g. WiFi, etc.) communication options. In some examples, the gateway may be configured to use the wired and/or wireless communication option when it is determined that cellular communication is not available. In some examples, the gateway may be configured to use the wired and/or wireless communication option when it is determined that the wired and/or wireless communication is preferred over cellular communication. For example, if it is determined that a cellular connection is not strong enough (e.g., a signal strength is not above a predetermined threshold).

In some embodiments, a wearable device may transmit pings. When a packet is received, the wearable device measures the strength of the signal (e.g., RSSI value or another suitable value). If several packets are received from a single device in a row that exceed a signal strength threshold, the wearable devices may indicate that its proximity to another wearable device is too close. In some embodiments, wearable devices may emit pings with a lower rate when no contacts are present, and at a higher rate when a possible contact is detected. Further, the signal strength threshold may also be predetermined or set by a user (e.g., using an interface). In some embodiments, the threshold signal strength is correlated with a distance between a first wearable sensor and at least one of the one or more wearable sensors attached to other users.

In some embodiments, the number of packets exceeding a signal strength threshold to trigger the indication may be a predetermined number included in the firmware. In some embodiments, the number may be set by a user of the system. In some embodiments, the number can be set by a user using an interface described herein.

In some embodiments, the triggered indication may include an issued alert (e.g., by flashing its red LED and/or producing an audible tone). In one example, the tone can progressively increase in intensity, starting with a simple reminder beep and progressing to a full siren tone if the wearable devices do not separate. In some embodiments, the wearable device includes a button on the wearable device that can act to silence the wearable device. In some embodiments, the wearable device can be configured to auto-sleep if motion is not detected for some time period. In some embodiments, the alerts for devices may be turned off for using the devices for contact tracing purposes only. In some embodiments, a wearer of the wearable device may be allowed to silence a predetermined number of alarms.

In some embodiments, in addition to the audible and visible proximity alerts, the wearable device is configured to record a history of other wearable devices that came within close range of the wearable device. The wearable device may record a timestamp and duration of the encounter. This data can be downloaded periodically or on demand to a Cloud dashboard using a cellular-connected Cloud Pod or another suitably connected gateway. For example, the interactions may be sent to cloud via gateway if an active gateway/network is within range.

In some embodiments, the cellular-connected Cloud Pod includes a processor, memory, and cellular interface for communicating cellular data, and a radio that allows it to receive data from individual wearable devices. One or more Cloud Pods may be placed near site exits to capture contact session data from wearable devices, e.g., as workers departed the site. Cloud Pods may also be placed in high-traffic areas of the site to gather data over the course of the workday. Cellular-connected Cloud Pods running special firmware can be placed near entrances or other key areas to receive and record the proximity of wearable devices, providing data about workers arriving, leaving, or congregating. In some embodiments, the Cloud Pod has a self-contained pre-activated cellular connection supporting customer self-deployment. The Cloud Pod device may be used for example, by simply hanging the Cloud Pod device on a wall and plugging it in. In some embodiments, a gateway comprises a cellular communication component, and the gateway may be adapted to communicate contact session data to the management system over the cellular communication component.

In some embodiments, a Cloud Pod may additionally or alternatively include a wired and/or wireless communication component. For example, a gateway may include a wired communication component (e.g. Ethernet, etc.), and may be adapted to communicate contact session data to the management system over the wired communication component. In some examples, a gateway includes a wireless communication component (e.g. WiFi, etc.), and may be adapted to communicate contact session data to the management system over the wireless communication component.

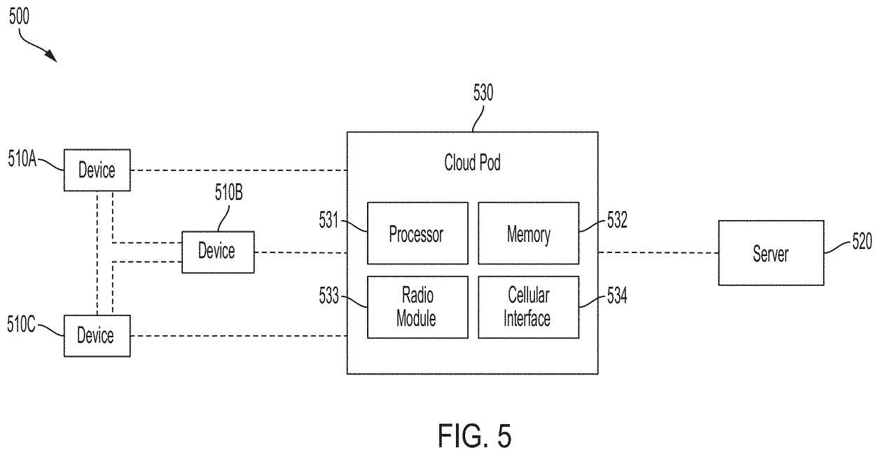

FIG. 5 is a diagram of exemplary system 500, according to some embodiments. The devices 510A-C may transmit data about contact sessions and/or the like to one or more servers 520 using a Cloud Pod 530. The Cloud Pod 530 may include a processor 531, a memory 532, a radio module 533 and a cellular interface 534.

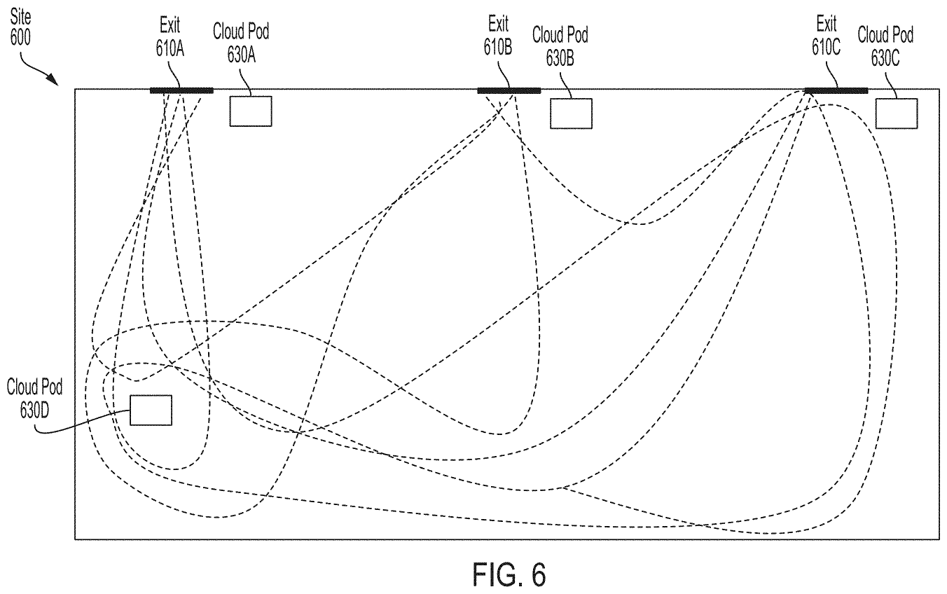

FIG. 6 shows an exemplary site 600, according to some embodiments. As described herein, Cloud Pods may be located near the site exits to capture contact session data from wearables, such as exemplary Cloud Pods 630A, 630B, and 630C which are located near exemplary exits 610A, 610B, and 610C. The dashed lines of FIG. 6 may represent traffic by workers and/or other users of the wearable devices. The Cloud Pod 630D is located in an area corresponding to high traffic on the site. Long-range cellular-enabled gateways (Cloud Pods) provide ease-of deployment (customer self-install) while enabling regular upload of contact data (avoids need to collect wearable from infected individual for contact tracing).

In some embodiments, full-site network coverage is not required. For example, if there are areas of a site where a device is not in range to communicate with any gateway, the device may store interactions (e.g., contact sessions) for later upload to the next available gateway (e.g., Cloud Pod) if no network is available.

In some embodiments, the wearable devices may be capable of communicating on a wireless mesh network, and may be operable to receive alert messages from different entities, such as sensors, management systems, communication devices (e.g., a supervisor mobile device), or other entities. For example, a wireless communication network may be configured on the worksite including one or more nodes that are interconnected within a mesh network. Further, the sensors may communicate over the mesh network by communicating with one or more nodes which relay the messages to a distributed computer system which may include one or more management interfaces used for the purpose of monitoring users, events, and their associated data.

In some embodiments, the system is capable of supporting a worker traveling between different areas. For instance, the wearable may be capable of identifying and joining a mesh network at any one of multiple geographically distinct locations. Upon joining any network, all of the features of the sensor may be available, along with identification of the site's network to which the sensor is connected. The system may be configured (e.g., using a management system) to associate the sensor with multiple mesh networks, and when the wearable device comes within a communication range of the network, it may automatically join the network.

In some embodiments, the system is capable of supporting a worker traveling between sites wherein events may be locally stored within the sensor along with location data. For instance, the wearable device may, according to one implementation, be capable of storing alerts detected when not connected to the mesh network, or alternatively, transmitting them through an alternate network (e.g., cell phone network, Bluetooth, or other communication capability). The sensor may also be configured to transition to an unconnected mode when not in range of the mesh network (or any other network).

In some embodiments, the wearable device may include one or more network interfaces through which the sensor device communicates information to other systems. To this end, the wearable device may also include one or more antennas that permit the sensor to communicate wirelessly to one or more mesh nodes within the mesh network.

FIG. 7 shows exemplary control flow of a wearable device. A device may be initialized at a starting state 7000 and enter a sleep mode 7010 automatically. The device may exit the sleep mode 7010 if motion has been detected. The device may enter sleep mode if motion has not been detected for a predetermined period of time (e.g., 10 seconds). If motion has been detected, the device may listen for pings and advertisements by other devices for a period of time at state 7020 (e.g., 100 ms or another suitable time period).

If the device receives a ping with an RSSI value determined to be higher than a threshold at 7030, the device records the other device's serial number and/or a timestamp in the contact session in state 7040. The device may then generate audible and visual alerts with progressive intensity over time in state 7050.

If the device does not receive a ping with an RSSI value determined to be higher than the threshold at state 7030, or after state 7050, the device checks for received advertisements at state 7060. If the device has received an advertisement, the device receives the configuration from the Cloud Pod in state 7070 and transmits open and closed contact sessions to the Cloud Pod in state 7080. The device then listens for acknowledgment or ACK from the Cloud Pod and marks ACK′d contact sessions (state 7090).

After state 7090 or if the device did not receive an advertisement in state 7060, the device may transmit one or more pings in 7100. In some embodiments, the device may transmit pings for a given period of time. In some embodiments, the device may transmit pings in intervals for a given period of time (e.g., 100 ms+/−10 ms for 900 ms). In some embodiments, the device may transmit pings a predetermined number of times.

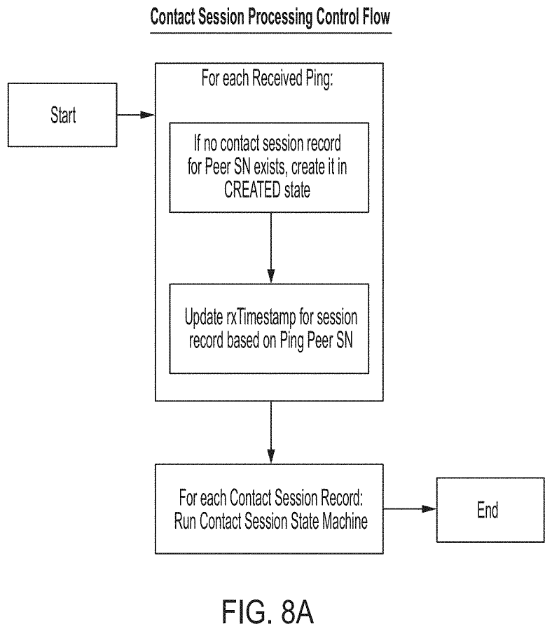

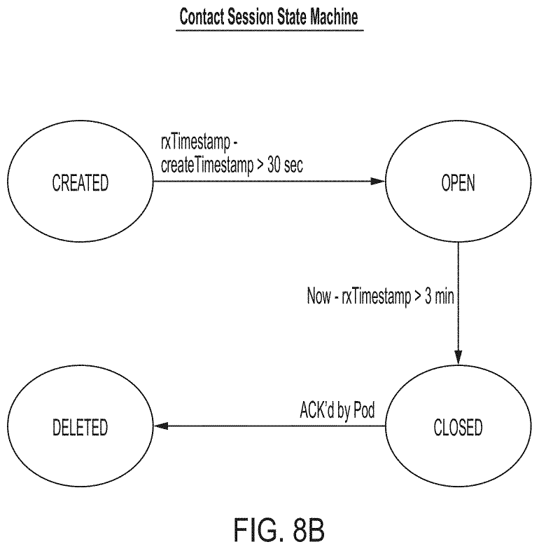

The device may then process the contact sessions in 7110, as described further in relation with FIGS. 8A and 8B. If the device does not detect motion for a predetermined period of time in state 7120 after state 7110, the device may then return to the sleep mode. Or, if motion has been detected, the device may listen for pings and advertisements in state 7020.

### Local Session Determination

In some aspects, methods and techniques described herein relate to locally determining a contact session of a device. In some embodiments, the system is adapted to convert pings, which are any instance where one device detects another, and related ping data, into contact sessions, which may indicate a period of sustained contact. In some embodiments, the system is adapted to transmit contact sessions, and not pings or ping data, to the server to reduce the amount of data that needs to be stored and transmitted. The ping data may include information corresponding to one or more pings. In some embodiments, a contact session minimally includes Peer Device ID, Start Timestamp, and Duration.

In some embodiments, a contact session with a peer device is opened any time one or more pings are received from that device for more than a certain amount of time (e.g., 30 seconds). In some embodiments, the system is designed to ignore very brief encounters, such as two people walking past each other. Once a session is open, its duration is tracked, and the duration is extended every time another ping is received from the same peer device. The session is closed when no ping has been received from that peer for more than a predetermined time (e.g., 3 minutes). Both open and closed sessions can be transmitted to the servers. In some embodiments, a closed session can be deleted from the wearable device when acknowledged, while an open session can be maintained and have its duration updated as additional pings are received.

In some embodiments, a wearable may be programmed to perform the following control functions as described herein. For example, FIG. 8A shows an exemplary contact session processing control flow as performed by a device. For each received ping, if a contact session record for the serial number associated with the peer device does not exist, the device may create the contact session in a CREATED state. The device may then update the timestamp corresponding to the received ping (e.g., rxTimestamp) for the session record based on the peer device's serial number. For each of the contact session records, the device may then run the contact session state machine described in relation with FIG. 8B.

As discussed, FIG. 8B shows an exemplary contact session state machine for a device, according to some embodiments. The device may be at a CREATED state. If the time elapsed between receiving and creating a timestamp exceeds a predetermined period of time (e.g., rxTimestamp−createTimestamp>30 sec), the device may enter an OPEN state. If the time elapsed between a current time and receiving timestamp exceeds a predetermined period of time (e.g., Now−rxTimestamp>3 minutes), the device may enter a CLOSED state. At the CLOSED state, if an acknowledgement from a Cloud Pod is received, the device may enter a DELETED state.

### Toggling Power Level for Transmissions

In some embodiments, the wearable device may communicate in a sub-GHz radio band. The sub-GHz radio band measuring device-to-device proximity provides more uniform ranging performance both indoors and outdoors and near human bodies relative to Bluetooth, GPS, and Long Range (LoRa) modulation techniques. A single sub-GHz radio used for both communications may provide very low-power for proximity and long range for uplink with consistent performance at low cost.

In some embodiments, the device may communicate in a high power mode and/or a low power mode. The high power mode may use more power than the lower power mode and may also increase communication range for the device further than the communication range corresponding to the lower power mode.

In some embodiments, when the device performs device-to-device communication, the device may communicate using a low power mode. In some embodiments, when a device performs an uplink to a gateway (e.g., Cloud Pod) the device communicates in a high power mode. The device may include a transmitter adapted to communicate with the one or more wearable sensors attached to other users using a sub-GHz radio band when in the low power mode. The device may include a transmitter adapted to communicate with the gateways using a sub-GHz radio band when in the high power mode. For example, the radio 320 of device 300 may include a transmitter adapted to communicate with other devices on users using a sub-Ghz radio band and/or a transmitter adapted to communicate with gateways using a sub-Ghz radio band.

Communicating to gateways in a high power mode allows the signal range of devices to be wider than for low power communications. This may decrease the need for more gateways, as devices are able to transmit from further away, and network coverage can be achieved with fewer gateways.

By communicating between devices using a low power mode, the power on the devices may be saved significantly. This may also prevent interference between devices that are further away such that communications occurring outside a device's communication range do not interfere with communications to and/or from the device.

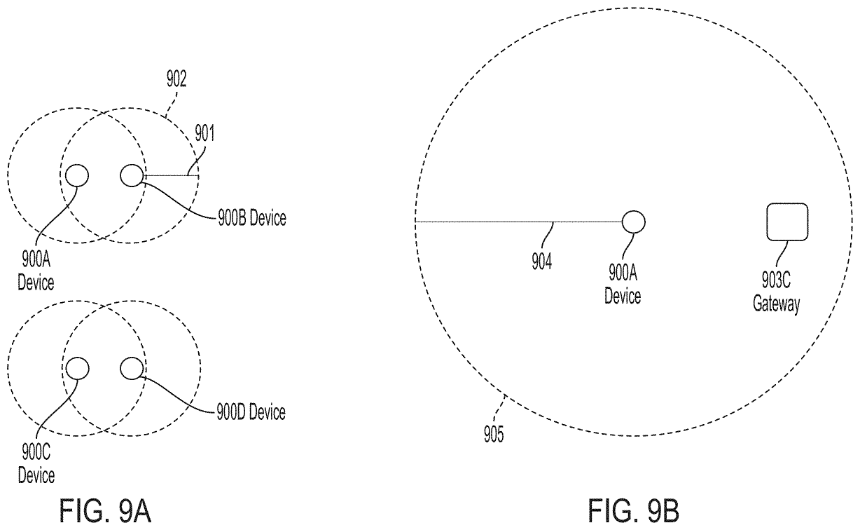

FIG. 9A and FIG. 9B are exemplary signal strength ranges of device-to-device communication using a low power signal and device-to-gateway communication using a high power signal. For example, in FIG. 9A, device 900A and 900B are communicating using a low power mode, which corresponds to a signal range 902, with radius 901. Devices 900C and 900D are communicating using a low power mode, which corresponds to a signal range 902, with radius 901. The signals of devices 900A and 900C do not interfere with each other as they are communicating to other devices in a low power mode.

FIG. 9B shows the device 900A during a high power mode device-to-gateway communication with gateway 903C. The device 900A is communicating in a high power mode and so the signal ranges further than for the low power communication of FIG. 9A. For example, the device 900A has a signal range of 904 having a radius of 905 during high power mode communications. Radius 905 of FIG. 9B is larger than the radius 901 of FIG. 9A.

In some embodiments, a single common channel used to detect proximity and advertise gateways (e.g., Cloud Pods). Devices may transmit for proximity at low power, gateways (e.g., Cloud Pods) may advertise at high power. In some embodiments, gateway (e.g., Cloud Pod) advertisements contain channel assignments to be used when communicating to that specific gateway. Gateways may periodically send site configuration on their specific channels, allowing configuration and tuning of thresholds and behaviors. Devices may send contact session data on one gateway-specific channel and receive acknowledgements on another, minimizing collisions and channel contention. This may be done at high transmit power to provide a long range.

In some embodiments, a device may be, by default, on a low power mode and may enter a high power mode to communicate with a gateway. In some embodiments, the device may enter a high power mode when a gateway (e.g., Cloud Pod) is detected, for example by gateway (e.g., Cloud Pod) advertisements.

### Accuracy

The inventors have conducted extensive testing and demonstrated that, in some aspects, measuring device-to-device distance accurately using a radio frequency (RF) solution may be challenging, however it can be achieved within certain bounds. RF signals propagate differently based on the environment, with reflections and interference causing some variability in received signal strength. RF parameters can be tuned to minimize these effects, and the inventors have tested a solution which varies little (e.g., <20%) between indoor and outdoor environments.

It is appreciated that the RF radiation pattern from the device is not uniformly omnidirectional, especially when being worn. This creates some variability in distance sensitivity based on direction relative to the device. With mounting to the front of the hardhat, the most sensitive zone is directly in front, with an approximate detection range of 6-10 feet.

The sides may be slightly less sensitive with a range of 4-8 feet, and directly behind may be the least sensitive zone due to the attenuation caused by the wearer's head, with detection range of 2-4 feet. This sensitivity is in an appropriate range to effectively give immediate real-time warnings about close contact as well as produce an accurate list of candidates for contact tracing.

In some embodiments, the system may use Automatic Transmitter Calibration techniques as described herein, to improve accuracy of ranging. For example, Cloud Pods may perform the Automatic Transmitter Calibration in order to obtain a calibration value of each Cloud Pod. In some embodiments, the wearable devices may also be configured to perform Automatic Transmitter Calibration (e.g., when a wearer of the wearable device is determined to not be moving).

In some embodiments, the system may use Multipath Interference Mitigation techniques to improve accuracy of ranging. For example, each wearable device may periodically transmit a message (e.g., a packet) containing a serial number identifier to identify the wearable device a few times per second and may also listen for identifier packets when not transmitting. When a packet is received, the wearable device may perform the Multipath Interference Mitigation techniques to determine a characteristic strength of the signal and use the characteristic strength of the signal to then determine and indicate if the wearable's proximity to another wearable device is too close.

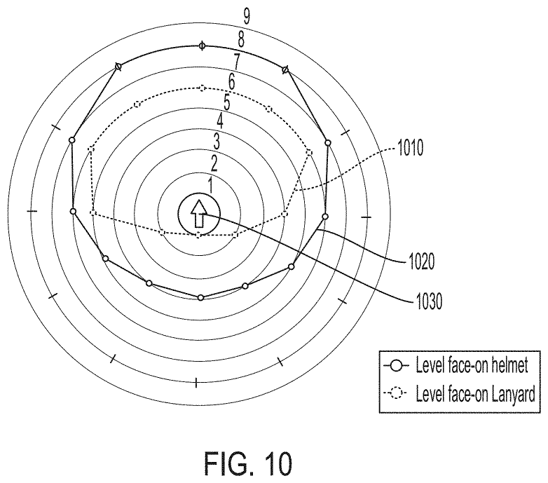

FIG. 10 shows the results of a performance test. Testing was performed using a modified firmware which would transmit packets several times per second and listen for packets the rest of the time, emitting an audible beep when packets were received above a set RSSI threshold. Tests were performed with a pair of devices mounted to hardhats, with the hardhats both on and off of a human test subject. A variety of RF parameters (transmit power, RSSI thresholds, modulation, etc.) in order to determine those with the most consistent performance across environments and relative orientations. Tests were also performed with the device on a lanyard worn around the neck. The range at which the beeping started was plotted for each test condition and angle.

In FIG. 10, the curve 1010 represents the location at which the device on the lanyard detected another device's packet with an RSSI value higher than the predetermined threshold when the wearer was facing in the direction of arrow 1030. In FIG. 10, the curve 1020 represents the location at which the device on the hardhat detected another device's packet with an RSSI value higher than the predetermined threshold when the wearer was facing in the direction of arrow 1030.

### Web Dashboard and Reporting

Various aspects of the systems and methods described herein relate to interfaces through which the user can interact with a management system to monitor subjects. The data from the wearable devices may be transmitted to the Cloud-based servers to be stored in a database (e.g., via a Cloud Pod, or through the mesh network as described herein). This data may include the wearable device serial number as well a list of contact sessions, each of which can contain a peer wearable serial number, the start time of a determined contact session, and a duration of the contact session.

As discussed, a cloud-based server may be capable of performing one or more management functions with the sensor-based system. Such management functions may include monitoring sensor devices and locations, viewing events, performing analysis of events, allocating sensors to individuals and pieces of equipment, monitoring employee performance, and monitoring equipment usage, among other functions. To this end, the cloud-based server may include one or more management interfaces to facilitate these functions.

For example, the interface may show a map layout of a particular workplace within a management interface presented to the user (e.g., an administrator). The interface may have the capability of mapping the approximate location of contact session or other interactions between wearers within certain designated areas of the workplace. An administrator may be capable of configuring zones and monitoring workers within those zones.

A web dashboard may provide a user with the ability to generate reports and may be accessed by specific users of the system. For example, the web dashboard may include secure login for both staff and Customers such that only users allowed access can utilize the web dashboard.

In some embodiments, the wearable devices may be used to gather data and generate reports in order to provide information relating to the movement of different workers. The web dashboard may correspond a worker to a serial number.

In some embodiments, a Daily Roster Report may be generated to list workers who were present on site on a given day and/or date range. The date range and/or day can be determined by the user of the web dashboard via a graphical interface.

In some embodiments, a Social Distancing report may be generated, which may provide information on workers sorted by highest accumulated contact time, and may be intended for coaching behavior modification (e.g., if a number of workers continue to exhibit a high amount of contact time).

In some embodiments, a Contact Tracing Report may be generated, which may provide information on workers who came into contact with a specified worker over a specified range of dates. This report may be sorted by highest total contact time, according to some embodiments. In some embodiments, the user of the interface/web dashboard may choose to omit workers with less than a specified amount of accumulated contact time over a period of time (e.g., option to omit workers with less than 15 minutes of contact per day). For example, per client request, the dashboard may be configured to run a contact tracing report (subject 1 candidates) and deliver an Excel packaged data file (requires infected individual's name or device serial identification and desired date range). The data may include one or more of timestamp, duration of interaction, and close interacting wearers.

The web dashboard and/or interface may also include support functions to review battery level and signal strength on Pods and battery level on wearables, etc.

In some embodiments, the dashboard may be a simplified version of the dashboard described in commonly owned U.S. Patent Application Publication No. 2017/0270462, providing headcount/attendance, worker profile management, device management and administration. In some embodiments, the dashboard may be a separate interface than one used, for example, with one or more other types of wearable devices. In some embodiments, the interface for the wearable device and for the other types of wearable devices may be one interface.

It should be appreciated that the system may include other management features, and the systems and methods described herein are not limited to these features. Also, it should be appreciated that any of these features may be used alone or in conjunction with any other features described herein.

### Programming Wearable Devices

In one implementation of the sensor-based system, as described herein, the wearable devices may be sealed at the factory and may be updated with new firmware/software either at a distribution facility or in the field.

In some embodiments, the wearables may be programmed or reprogrammed using Over-The-Air programming using the accelerometer-based unlock sequence as described herein. For example, the wearable may be configured to determine if an acceleration measured by the accelerometer sensor indicates a programming or reprogramming mode. In some embodiments, if the wearable is placed in a particular orientation, and if that orientation is detected, the wearable may search for a signal from a programming device. The orientation may be detected, for example, responsive to a wake-up event, such as movement, an outside signal, or other activity. When such a signal is detected, the programming process is started from the programming device. In some embodiments, the signal may indicate whether the wearable should be programmed or reprogrammed to perform the methods described herein.

### Automatic Transmitter Calibration

Various aspects of the systems and methods described herein relate to estimating a distance between sensors and nodes. In some embodiments, the distance can be estimated using, at least in part, RSSI-based ranging. The inventors have recognized that transmitter output power varies from unit to unit by some dBm, while receiver accuracy of RSSI varies around 0.5 dBm, which may impact the accuracy of RSSI-based ranging. As such, some embodiments described herein relate to improving distance estimation by calibrating an RSSI value.

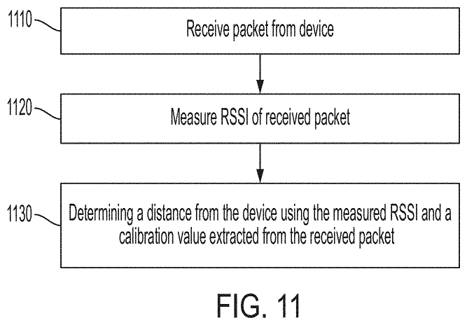

In some embodiments, a device (such as a Gateway or Tag device described herein) may store a calibration value. In some embodiments, the calibration value may be stored in non-volatile memory of the device and may be stored as a signed integer. The calibration value can be determined using methods described herein, including as described further in relation to FIGS. 11, 12, 13 and 14.

In some embodiments, the calibration value may be included in transmitted packets by a transmitting device. A receiving device may add the calibration value from a received packet to a measured RSSI value to determine a compensated RSSI value. The compensated RSSI value may be used to determine a range and/or estimated distance between the receiving and transmitting device. For example, the compensated RSSI value may be compared to different threshold values to determine a range or estimated distance.

FIG. 11 shows a flowchart of an exemplary process of determining distance using a calibration value. A receiving device may receive a packet from a transmitting device at step 1110. The receiving device may measure the RSSI value of the received packet at step 1120 and determine a distance from the device using the measured RSSI value and a calibration value of the transmitting device extracted from the received packet at step 1130.

In some embodiments, determining a distance may include adding the calibration value to the measured RSSI value, where the calibration value is a signed value.

Some aspects of the systems and methods described herein relate to a method of calibrating a device. The method can include a device being calibrated transmitting a calibration request to a calibration reference. The reference device may measure the RSSI value of the received calibration request and transmit the RSSI value of the received calibration request as well as the calibration value of the reference device. In turn, the device being calibrated may measure the RSSI value of the response packet and computes a value using the received values and the measured RSSI value of the response packet. For example, the value may be the RX-TX delta and may be calculated using the following formula:

RX_TX_DELTA=RESPONSE_RSSI−REQUEST_RSSI+REFERENCE_CALIBRATION

Wherein the RESPONSE_RSSI may be the RSSI value of the response packet as measured by the device being calibrated, the REQUEST_RSSI may be the RSSI value of the calibration request as measured by the reference device, and the REFERENCE_CALIBRATION is a calibration value of the calibration reference device.

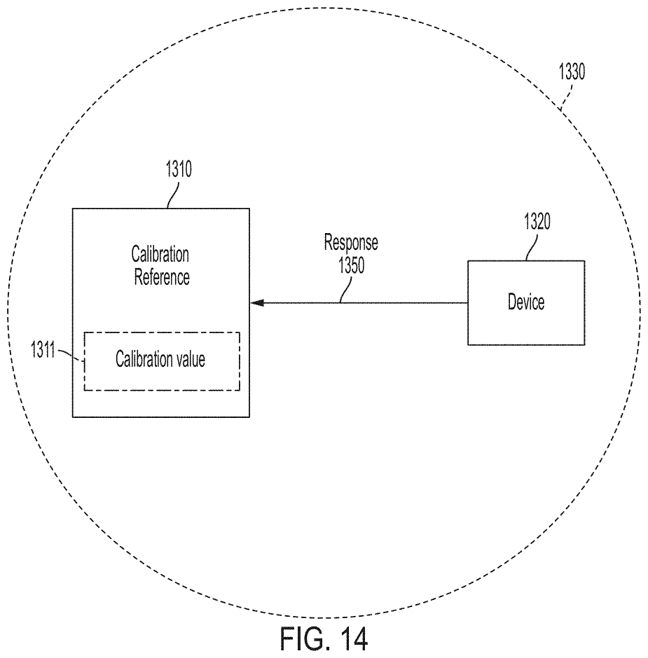

FIGS. 13 and 14 show an exemplary Calibration Reference 1310 and Device 1320. The Calibration Reference 1310 may be an already-calibrated gateway and the Device 1320 may be any device (e.g., tag device, gateway, etc.) to be calibrated. The Calibration Reference 1310 may comprise a calibration value 1311. In FIGS. 13 and 14, the Calibration Reference 1310 is determined to be within a close proximity to the Device 1320 to be calibrated. For example, distance 1330 may represent a boundary inside which a reference is determined to be within close proximity, and this boundary can be chosen based on an RSSI value determined by the device, and/or a distance determined from the RSSI value.

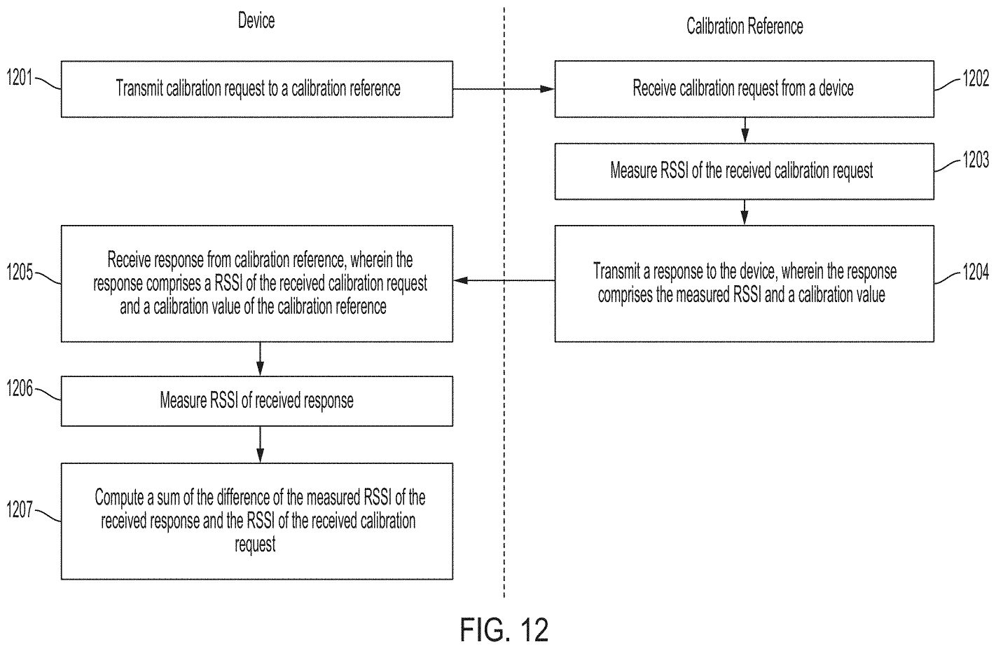

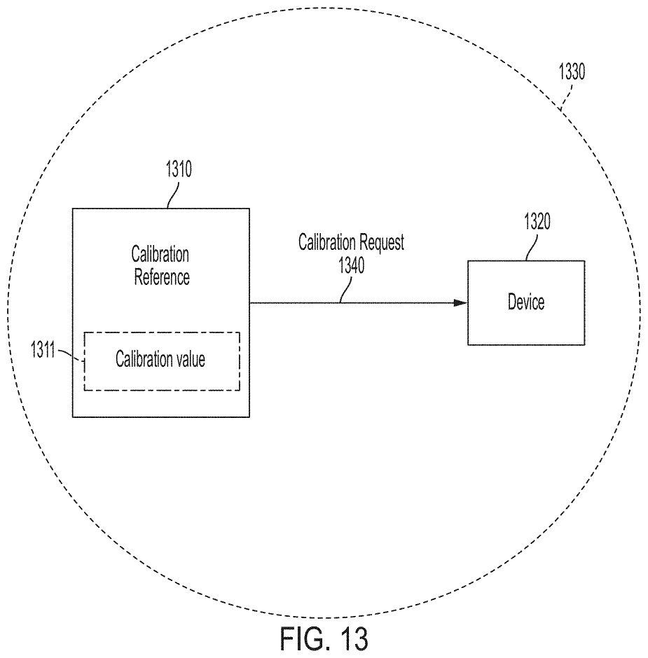

FIG. 12 shows a flowchart of an exemplary process performed by a device and a calibration reference device to calibrate the device. The process may include a device being calibrated transmitting a calibration request to a calibration reference at step 1201. The reference device may receive the calibration request 1340 at step 1202 and measure the RSSI value of the received calibration request at step 1203. The reference device may transmit the measured RSSI value of the received calibration request as well as the calibration value of the reference device in step 1204. The device may receive the response from the calibration reference. In some embodiments, the response comprises an RSSI value of the received calibration request and a calibration value of the calibration reference in step 1205. In turn, the device being calibrated may measure the RSSI value of the response packet (e.g., step 1206) and compute a value using the received values and the measured RSSI value of the response packet (e.g., step 1207).

The criteria may include, for example, the Calibration Reference 1310 being available and/or within close proximity of the Device 1320 to be calibrated. The close proximity may be determined for example using an RSSI threshold. In some embodiments, the criteria may include the Device 1320 requiring calibration. For example, the Device 1320 may be determined to require calibration based on an indication that the device has not been calibrated, some period of time having elapsed since a previous calibration of the device, and/or the like. In FIG. 13 and FIG. 14, the Device 1320 may not have a calibration value, and/or the calibration value may be outdated. The calibration reference may receive a response 1350 from the device. As described herein, the response 1350 comprises an RSSI value of the received calibration request and a calibration value of the calibration reference.

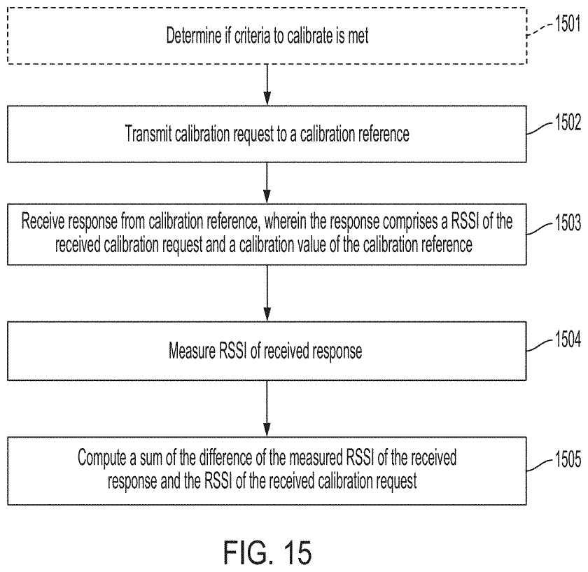

FIG. 15 shows a flowchart of an exemplary process for a device being calibrated. The process may include a device being calibrated transmitting a calibration request to a calibration reference at step 1502. The method may also include the device being calibrated measuring the RSSI value of the response packet (e.g., step 1503) and compute a value using the received values and the measured RSSI value of the response packet (e.g., step 1504). The method also includes computing a sum of the difference of the measured RSSI value of the received response and the RSSI value of the received calibration request in step 1505. The method may also have an optional step 1501 of determining if criteria to calibrate has been met. The criteria may include, for example, the Calibration Reference 1310 being available and/or within close proximity of the Device 1320 to be calibrated. The close proximity may be determined for example using an RSSI threshold. In some embodiments, the criteria may include the Device 1320 requiring calibration. For example, the Device 1320 may be determined to require calibration based on an indication that the device has not been calibrated, some period of time having elapsed since a previous calibration of the device, and/or the like.

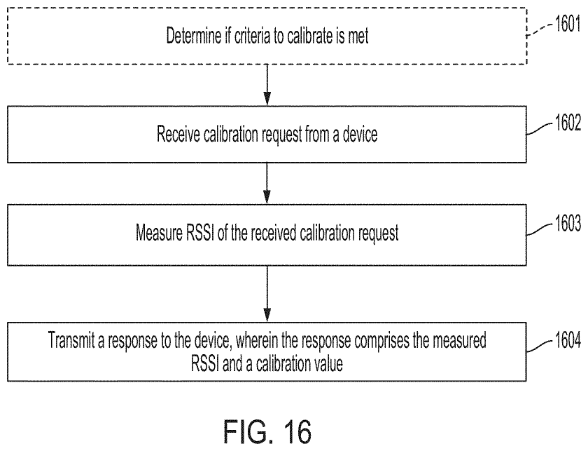

FIG. 16 shows a flowchart of an exemplary process for a calibration reference device. The reference device may receive the calibration request at step 1602 and measure the RSSI value of the received calibration request at step 1603. The reference device may transmit the measured RSSI value of the received calibration request as well as the calibration value of the reference device in step 1604. The method may also have an optional step 1601 of determining if criteria to calibrate has been met. The criteria may include, for example, the Calibration Reference 1310 being available and/or within close proximity of the Device 1320 to be calibrated. The close proximity may be determined for example using an RSSI threshold. In some embodiments, the criteria may include the Device 1320 requiring calibration. For example, the Device 1320 may be determined to require calibration based on an indication that the device has not been calibrated, some period of time having elapsed since a previous calibration of the device, and/or the like.

In some embodiments, the value (e.g., RX_TX_DELTA) calculated in this way may be stored and used as the calibration value for that device. According to other embodiments, the value may be stored, and the method of FIGS. 11, 12, and 15 may be repeated a number of times (e.g., 10, 20, 50, etc.). The stored values may be then used to determine the device's Calibration Value, for example, by averaging the stored values, by taking a median of the stored values, etc.

In some embodiments, automatic transmitter calibration may be used with multipath interference mitigation as described herein. For example, when transmitting and receiving RSSI values, the devices may user multipath interference mitigation to get a characteristic RSSI value rather than one RSSI value determined using one channel.

In some embodiments, automatic transmitter calibration may be used with devices and Cloud Pods. For example, Cloud Pods may act as either Calibration References or devices to be calibrated. For example, a device or gateway (e.g., Cloud Pod) that has been determined to fulfil the criteria to calibrate may transmit a calibration request to a calibration reference (e.g., device, gateway, etc.). The calibration reference may then measure the strength (e.g., RSSI value) of the request and transmit the measured RSSI value along with the reference's calibration value. The device or gateway being calibrated may then measure the strength of the received packet and use this new measurement and the measured RSSI value to determine a calibration value.

In some embodiments, a first device may transmit a calibration value of the device as part of the device-to-device communication. A second device receiving the packet may measure the RSSI value of the packet and add the calibration value from the packet transmitted by the first device.

In some embodiments, automatic transmitter calibration may be used with a mesh network. For example, nodes may act as either Calibration References or devices to be calibrated.

In some embodiments, a sensor/device/wearable that has been determined to be still (e.g., using accelerometer, etc.) may be calibrated while the sensor/device/wearable continues to be determined to be still. In some embodiments, a user may be prompted to stay still so a sensor/device/wearable can be calibrated.

### Multipath Interference Mitigation

Some aspects of the systems and methods described herein relate to estimating a distance between sensors and nodes. In some embodiments, the distance can be estimated using, at least in part, RSSI-based ranging. Multipath Interference occurs when radio waves from a transmitter follow multiple reflected paths to a receiver, causing constructive or destructive interference at the location of the receiver which in turn is measured as a change in RSSI and may have a significant impact on RSSI-based ranging accuracy. Because the location of the interference nodes is based on wavelength of the radio wave, and wavelength is inversely proportional to frequency, the locations of interference nodes may change based on the frequency of the radio signal.

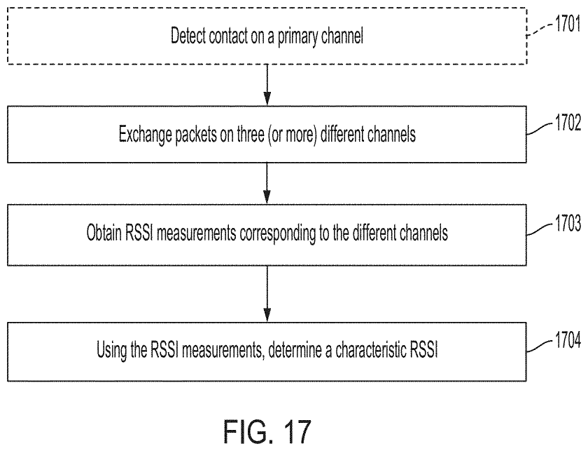

Some aspects relate to mitigating the effects of multipath interference. FIG. 17 is a flowchart of an exemplary multipath interference mitigation method.

A device may perform a multipath interference mitigation method in response to a contact detected on a primary channel, such as can be seen in step 1701. A contact may be detected, for example, via receipt of a ping packet. A primary channel could be a predetermined channel or another suitably selected channel. In some embodiments, a primary channel could be determined automatically to be the channel that provides a maximum, minimum or median RSSI value.

The multipath interference mitigation method may include a device exchanging packets on three or more different channels at step 1702. In some embodiments, the channels may be located at a low-end, middle and high-end of an available frequency band.

The device may obtain three RSSI measurements corresponding to the received packets of the three different channels at step 1703. The device may use the three RSSI measurements to determine a characteristic RSSI value at step 1704.

In some embodiments, the characteristic RSSI value may be determined by averaging the three RSSI measurements. In some embodiments, the characteristic RSSI value may be determined by averaging the two highest RSSI measurements of the three RSSI measurements. In some embodiments, the characteristic RSSI value may be a minimum RSSI value or a maximum RSSI value. In some embodiments, the characteristic RSSI value may be a median RSSI value. In some embodiments, the characteristic RSSI value may be an average of the two lowest RSSI values.

The characteristic RSSI value may be used for downstream processing in lieu of a single RSSI measurement. Some methods and techniques described herein may be performed using a characteristic RSSI value determined using the above described methods.

In some embodiments, multipath interference mitigation may be used with calibration values determined using automatic transmitter calibration. For example, when the device obtains three RSSI measurements corresponding to the received packets of the three different channels (e.g., at step 1703), the device may add the calibration value to each of the RSSI measurements to get calibrated RSSI values. The device may then use the three calibrated RSSI measurements to determine a characteristic RSSI value (e.g., at step 1704). In another example, the device may instead add the calibration value to the characteristic RSSI value.

In some embodiments, a device may use multipath interference mitigation to determine the RSSI value used in determination of whether another device is within a predetermined distance.

Having thus described several aspects of at least one embodiment, it is to be appreciated various alterations, modifications, and improvements will readily occur to those skilled in the art. Such alterations, modifications, and improvements are intended to be part of this disclosure, and are intended to be within the spirit and scope of the systems and methods described herein. Accordingly, the foregoing description and drawings are by way of example only.

What is claimed is:

1. A system comprising:
a first wearable sensor configured to be attached to a first user, the first wearable sensor adapted to periodically transmit a message, the message containing a sensor identifier that uniquely identifies the first wearable sensor, wherein the first wearable sensor is configured to listen for one or more messages transmitted by one or more wearable sensors attached to other users, and wherein the first wearable sensor is adapted to determine a signal strength of a received message, and based on determining that the signal strength is greater than a threshold, issue an alert; and
a gateway adapted to communicate with the first wearable sensor.

2. The system according to claim 1, wherein the first wearable sensor is configured to communicate with one or more wearable sensors attached to other users in a low power mode, and wherein the first wearable sensor is configured to switch to a high power mode in order to communicate with the gateway, the high power mode using more power than the lower power mode and increasing communication range for the first wearable sensor.

3. The system according to claim 2, wherein the first wearable sensor comprises a transmitter adapted to communicate with the one or more wearable sensors attached to other users using a sub-GHz radio band when in the low power mode.

4. The system according to claim 1, wherein the gateway is adapted to capture contact session data from one or more wearable sensors including the first wearable sensor.

5. The system according to claim 1, wherein the first wearable sensor is adapted to generate contact session data, the contact session data identifying at least one contact session set of information comprising a time at which the contact occurred, a duration of the contact, and an identifier of a sensor which came into proximity of the first wearable sensor.

6. The system according to claim 5, wherein the first wearable sensor generating the contact session data includes the first wearable sensor converting ping data from the one or more wearable sensors attached to other users into the contact session data, including the at least one contact session set of information comprising the time at which the contact occurred, the duration of the contact, and the identifier of the sensor which came into proximity of the first wearable sensor.

7. The system according to claim 6, wherein transmitting the contact session data to the gateway uses less bandwidth than transmitting the ping data from the one or more wearable sensors attached to other users.

8. The system according to claim 1, wherein the first wearable sensor and the gateway each store a calibration value to include in a packet, and wherein a receiving device adds the calibration value from the packet to a measured Received Signal Strength Indicator (RSSI) value for the packet to determine a compensated RSSI value for the packet, the compensated RSSI value being compared to one or more thresholds to determine range.

9. The system according to claim 8, wherein the gateway serves as a calibration reference device, and wherein the calibration value of the first wearable device is determined by:
transmitting, from the first wearable device, a calibration request to the calibration reference device;
measuring, at the calibration reference device, an RSSI value of the calibration request;
transmitting, from the calibration reference device, a response including the measured RSSI value of the calibration request and a calibration value of the calibration reference device;
measuring, at the first wearable device, an RSSI value of the response; and
computing, at the first wearable device, the calibration value of the first wearable device using the RSSI value of the response, the RSSI value of the calibration request, and the calibration value of the calibration reference device.

10. The system according to claim 1, wherein the first wearable device and the gateway are configured to perform multipath interference mitigation, including:
exchanging packets on three or more different channels located at a low-end, middle and/or high-end of a frequency band;
obtaining three or more RSSI measurements corresponding to each of the three or more different channels; and
determining a characteristic RSSI value based on the three or more RSSI measurements.

11. The system according to claim 1, wherein the first wearable sensor is adapted to sound an audible alert.

12. The system according to claim 11, wherein the first wearable sensor is adapted to sound a progressive alert based at least in part on the duration of proximity to one or more wearable sensors attached to other users.

13. The system according to claim 1, wherein the first wearable sensor is adapted to attach to a hard hat of the first user.

14. The system according to claim 1, wherein the first wearable sensor is configured to attach to a lanyard of the first user.

15. The system according to claim 1, wherein the sensor comprises an alert indicator and is adapted to activate the indicator in response to determining that the signal strength exceeded the threshold.

16. The system according to claim 1, further comprising a cloud-based storage system for storing contact session information communicated by the first wearable sensor.

17. The system according to claim 1, wherein the threshold signal strength is correlated with a distance between the first wearable sensor and at least one of the one or more wearable sensors attached to other users.

18. A system comprising:
a plurality of wearable sensors configured to be attached to respective ones of a plurality of users;
at least one gateway adapted to capture contact session data from the plurality of wearable sensors, the contact session data including contact events that identify contact that has occurred between respective pairs of the plurality of wearable sensors; and
a management system adapted to store the contact session data from the plurality of wearable sensors.

19. The system according to claim 18, wherein each one of the plurality of wearable sensors are adapted to record contact session data, the data identifying at least one contact session set of information comprising a time at which the contact occurred, a duration of the contact, and an identifier of a different wearable sensor which came into a proximity of at least one of the plurality of wearable sensors.

20. The system according to claim 18, wherein each one of the plurality of wearable sensors comprises a transmitter adapted to communicate messages using a sub-GHz radio band.

21. The system according to claim 20, wherein each of the plurality of wearable sensors communicates with other ones of the plurality of wearable sensors via the sub-GHz radio band.

22. The system according to claim 21, wherein each of the plurality of wearable sensors communicates with the gateway using the sub-GHz radio band.

23. The system according to claim 18, wherein the gateway comprises a cellular communication component, and wherein the gateway is adapted to communicate contact session data to the management system over the cellular communication component.

24. The system according to claim 18, wherein the plurality of wearable sensors do not include GPS capability.

25. The system according to claim 18, wherein the plurality of wearable sensors do not include cellular communication capabilities.

26. The system according to claim 18, wherein the gateway comprises a wired communication component, and wherein the gateway is adapted to communicate contact session data to the management system over the wired communication component.

27. The system according to claim 18, wherein the gateway comprises a wireless communication component, and wherein the gateway is adapted to communicate contact session data to the management system over the wireless communication component.
